(19) United States
(12) Patent Application Publication
(10) Pub. No.: US 2012/0316589 A1
(43) Pub. Date: Dec. 13, 2012

(54) Balloon catheter with raised elements and visual marker

(75) Inventor: Darin Schaeffer, Bloomington, IN (US)

(73) Assignee: COOK MEDICAL TECHNOLOGIES LLC, Bloomington, IN (US)

(21) Appl. No.: 13/491,095

(22) Filed: Jun. 7, 2012

Related U.S. Application Data
(60) Provisional application No. 61/494,241, filed on Jun. 7, 2011.

(51) Int. Cl. A61B 17/24

(52) U.S. Cl. 606/170

(57) ABSTRACT

A balloon catheter has an elongate catheter shaft and an inflatable balloon. A marking indicia extends along the axial length or a portion of the axial length of the shaft. A raised element is formed by or disposed on the inflatable balloon and is aligned with the marking indicia. Methods of treating a portion of a bodily passage using a balloon catheter having a raised element on the inflatable balloon are also described.

### FIELD

The disclosure generally relates to the field of medical devices. Exemplary embodiments relate to balloon catheter assemblies having visual markers, and methods of using the same to treat a portion of a bodily passage.

### BACKGROUND

Caregivers frequently treat stenosis of bodily passages by expanding the passage from within using a balloon catheter. For example, balloon catheters are frequently used in the treatment of blood vessel stenosis and of airway stenosis. In these procedures, a caregiver typically navigates a balloon catheter through the stenosed passage until the balloon is positioned near or directly adjacent the stenosis. The balloon is inflated to force dilation of the passage at the stenosis. Multiple sequences of inflation and deflation of the balloon can be used to achieve the desired dilation.

Some dilation procedures present additional challenges. For example, airway stenoses—a narrowing of the pulmonary airway resulting in shortness of breath (dyspnoea), high-pitched noise (stridor), wheezing, hoarseness, and/or respiratory distress—often present with scar tissue in the airway that has formed in response to coughing and other consequences of the stenosis. When dilating an airway stenosis, a caregiver may need to cut or score scar tissue from the interior of the airway to fully dilate the airway at the stenosis.

Unfortunately, conventional cutting balloons can be ineffective for use in these airway dilation procedures on eccentric lesions, such as lesions in which scar tissue is localized to one side of the airway. Existing cutting balloons do not allow the caregiver to specifically direct the raised elements of the balloon to the area of the lesion that needs to be cut—the scar tissue. The raised elements contact the airway tissue at arbitrary points and, as a result, healthy tissue may be cut or scored while the scar tissue goes unaltered. This may ultimately lower the effectiveness of the dilation procedure.

A need exists, therefore, for improved catheters that facilitate scoring and/or cutting of tissue from within a bodily passage.

### BRIEF SUMMARY

Dilation catheters having an inflatable balloon, a raised element, and a visual marker are described.

An exemplary balloon catheter assembly comprises an elongate shaft, a marking indicia, an inflatable balloon, and a raised element. The elongate shaft has a proximal end, a distal end, a lengthwise axis, an exterior surface, a circumference, and an axial length and defines an inflation lumen. The axial length extends along the lengthwise axis from the proximal end to the distal end. The marking indicia is disposed on the elongate shaft and extends axially along at least a portion of the axial length of the elongate shaft. The inflatable balloon is attached to the distal end of the elongate shaft and cooperatively defines an inflation chamber with a portion of the exterior surface of the elongate shaft. The inflation chamber is in fluid communication with the inflation lumen. The inflatable balloon has deflated and inflated configurations and is adapted to move from the deflated configuration to the inflated configuration as fluid moves into the inflation chamber from the inflation lumen. The raised element is disposed on the inflatable balloon and aligned with the marking indicia. The marking indicia is adapted to indicate the location of the raised element about the circumference of the elongate shaft.

Another exemplary balloon catheter assembly comprises an elongate shaft, a marking indicia, an inflatable balloon, and a raised element. The elongate shaft has a proximal end, a distal end, a lengthwise axis, an exterior surface, a circumference, and an axial length and defines an inflation lumen. The axial length extends along the lengthwise axis from the proximal end to the distal end. The marking indicia is disposed on the elongate shaft and extends axially along at least a portion of the axial length of the elongate shaft. The inflatable balloon is attached to the distal end of the elongate shaft and cooperatively defines an inflation chamber with a portion of the exterior surface of the elongate shaft. The inflatable balloon has an outer surface, a proximal end, a distal end, and an axial length. The outer surface defines a balloon arc that extends between a proximal end point on the proximal end of the balloon and a distal endpoint on the distal end of the balloon. The inflation chamber is in fluid communication with the inflation lumen. The inflatable balloon has deflated and inflated configurations and is adapted to move from the deflated configuration to the inflated configuration as fluid moves into the inflation chamber from the inflation lumen. The raised element is disposed on the inflatable balloon and aligned with the marking indicia. At least one of the proximal endpoint and the distal endpoint is disposed adjacent the marking indicia. The marking indicia is adapted to indicate the location of the raised element about the circumference of the elongate shaft.

Methods of treatment utilizing a balloon catheter are also described.

An exemplary method comprises a method of treating a portion of a bodily passage. An initial step comprises inserting a catheter into said bodily passage. The catheter comprises an elongate shaft, a marking indicia, an inflatable balloon, and a raised element. The elongate shaft has a proximal end, a distal end, a lengthwise axis, an exterior surface, a circumference, and an axial length and defines an inflation lumen. The axial length extends along the lengthwise axis from the proximal end to the distal end. The marking indicia is disposed on the elongate shaft and extends axially along at least a portion of the axial length of the elongate shaft. The inflatable balloon is attached to the distal end of the elongate shaft and cooperatively defines an inflation chamber with a portion of the exterior surface of the elongate shaft. The inflation chamber is in fluid communication with the inflation lumen. The inflatable balloon has deflated and inflated configurations and is adapted to move from the deflated configuration to the inflated configuration as fluid moves into the inflation chamber from the inflation lumen. The raised element is disposed on the inflatable balloon and aligned with the marking indicia. Another step comprises advancing the catheter through the bodily passage with the balloon in an uninflated configuration until the uninflated balloon reaches an intended point of treatment within said bodily passage. Another step comprises confirming the orientation of the catheter relative to the portion of the bodily passage intended to be treated. Another step comprises positioning the catheter to align the raised element with the portion of the bodily passage intended to be treated by the catheter. Another step comprises inflating the inflatable balloon of the catheter until the raised element contacts the tissue of the portion of the bodily passage intended to be treated by the catheter. Another step comprises deflating the inflatable balloon. The steps of inflating and deflating the balloon can be successively repeated to achieve a desired number of repetitions, disruptions, and/or quality of disruptions in the surface of the tissue being treated. Another step comprises withdrawing the balloon catheter from the bodily passage. The marking indicia is adapted to indicate the location of the raised element about the circumference of the elongate shaft.

Additional understanding of the devices and methods contemplated and/or claimed by the inventor can be gained by reviewing the detailed description of exemplary devices and methods, presented below, and the referenced drawings.

### BRIEF DESCRIPTION OF THE DRAWINGS

FIG. 1 is a perspective view of a first exemplary catheter.

FIG. 2 is a side view of the exemplary catheter illustrated in FIG. 1.

FIG. 3 is a cross sectional view of the exemplary catheter illustrated in FIG. 1.

FIG. 4 is an end view of the exemplary catheter illustrated in FIG. 1.

FIG. 5 is a diagram depicting the inflatable balloon of the exemplary catheter illustrated in FIG. 1.

FIG. 6A is a perspective, environmental view of a second exemplary catheter.

FIG. 6B is a perspective, environmental view of the exemplary catheter illustrated in FIG. 6A.

FIG. 6C is a perspective, environmental view of the exemplary catheter illustrated in FIG. 6A.

FIG. 6D is a perspective, environmental view of the exemplary catheter illustrated in FIG. 6A.

FIG. 7 is a flowchart illustrating a first exemplary method of treatment.

FIG. 8 is a flowchart illustrating a second exemplary method of treatment.

### DETAILED DESCRIPTION

The following description and the referenced drawings provide illustrative examples of that which the inventor regards as his invention. As such, the embodiments discussed herein are merely exemplary in nature and are not intended to limit the scope of the invention, or its protection, in any manner. Rather, the description and illustration of these embodiments serve to enable a person of ordinary skill in the relevant art to practice the invention.

The use of “e.g.,” “etc,” “for instance,” “in example,” “or” and grammatically related terms indicates non-exclusive alternatives without limitation, unless otherwise noted. The use of “including” and grammatically related terms means “including, but not limited to,” unless otherwise noted.

The use of the articles “a,” “an” and “the” are meant to be interpreted as referring to the singular as well as the plural, unless the context clearly dictates otherwise. Thus, for example, reference to “marking indicia” includes the singular “marking indicium,” reference to “raised element” includes two or more such “raised elements,” and the like.

As used herein, the term “exemplary” means “an example of” and is not intended to convey a meaning of an ideal or preferred embodiment.

As used herein, the term “bodily passage” refers to any passage within the body of an animal. The term “bodily passage” includes elongate passages, such as the pulmonary airway, and cavities, such as sinus cavities.

The terms “score,” “scoring,” “cut,” and “cutting” refer to the making of disruptions in the surface of a tissue or a portion of a tissue, such as scar tissue, unless the context clearly dictates otherwise.

The terms “dilation” and “dilatation” are used interchangeably and refer to an enlargement or expansion in bulk or extent, the opposite of contraction, unless the context clearly dictates otherwise. Unless the context clearly dictates otherwise, usage of “dilation”/“dilatation” can include “score”/“scoring” and/or “cut”/“cutting.”

The term “aligned” and grammatically related terms refers to how a first structural element or feature is oriented with respect to a second structural element or feature. For example, “axially aligned” refers to an orientation in which an axis of each structural element or feature being described is disposed on a plane. Furthermore, “circumferentially aligned” refers to an orientation in which an axis of a first structural element or feature being described is disposed on a first plane and an axis of a second structural element or feature being described is disposed on a second plane that intersects the first plane at a defined angle (e.g., 90 degrees).

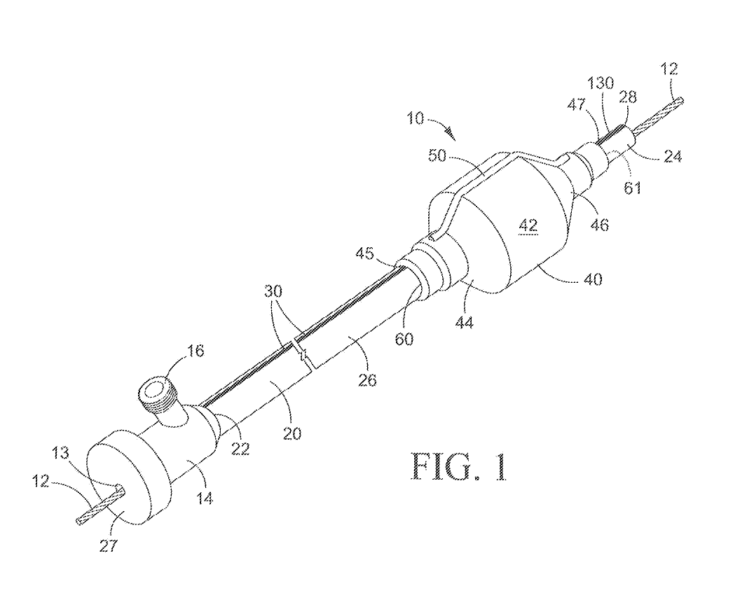

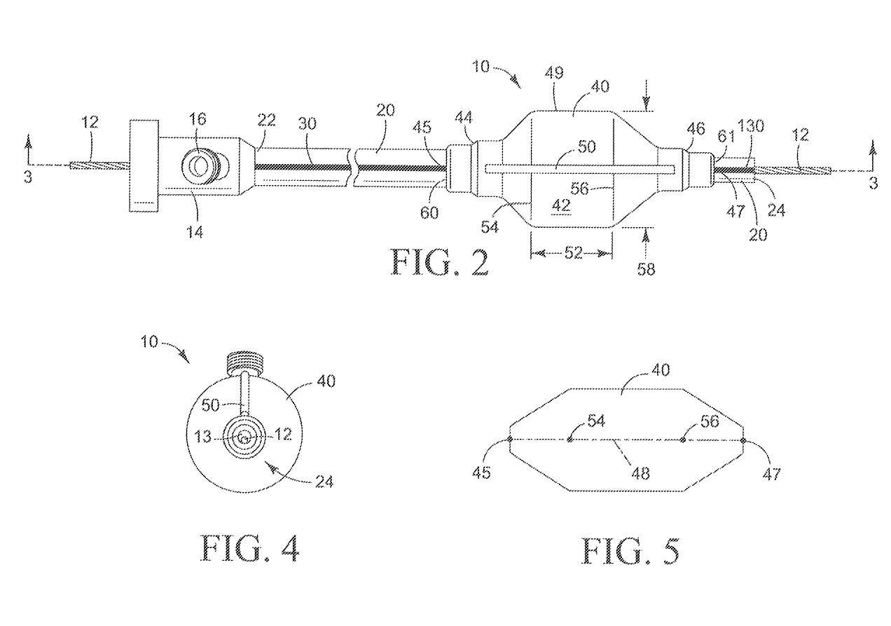

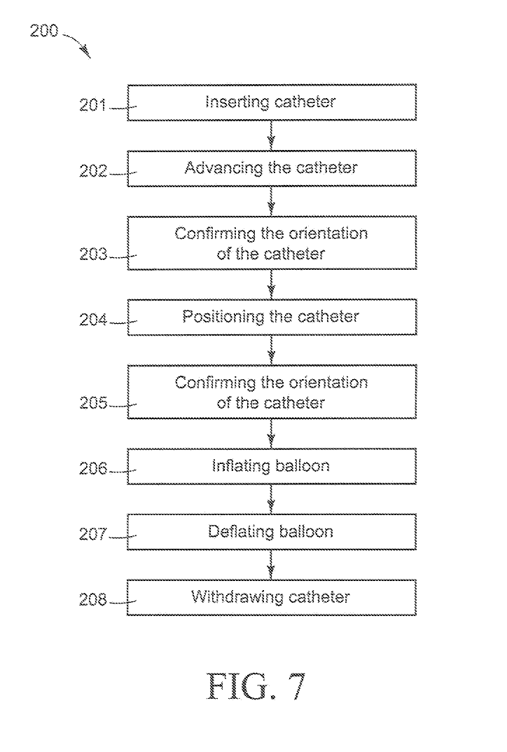

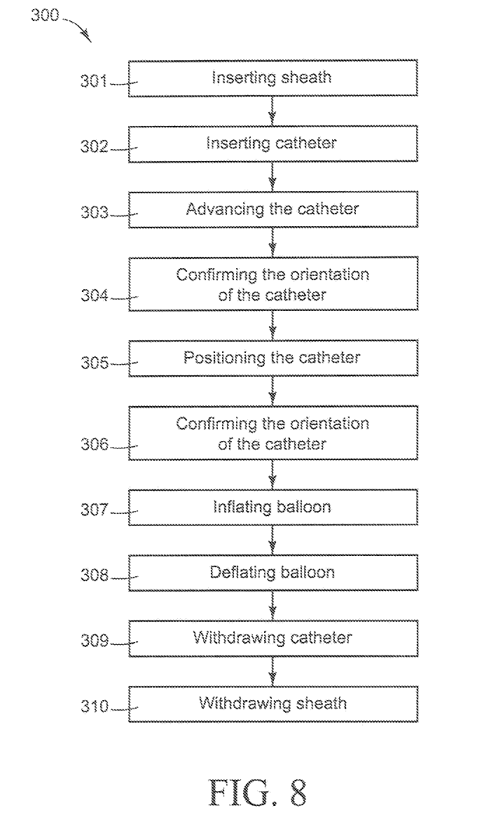

Exemplary balloon catheter assemblies are illustrated in FIG. 1 through FIG. 6D. Exemplary methods of dilating a bodily passage are illustrated in FIGS. 7 and 8.

FIG. 1 through FIG. 5 illustrate a first exemplary catheter 10. The catheter 10 includes an elongate shaft 20 extending between proximal portion 22 and distal portion 24. The shaft 20 defines an inflation lumen 18 and a wireguide lumen 13 and has a circumference. The inflation lumen 18 extends between an inflation port 16 and an opening 41 positioned on the distal portion 24 of the shaft 20. The wireguide lumen 13 extends from the proximal portion 22 to the distal portion 24 of the shaft 20.

Any suitable material can be used for the shaft 20 and other elements of the catheter 10, and skilled artisans will be able to select appropriate materials for a particular catheter based on various considerations, such as the nature of the bodily passage within which the catheter 10 is intended to be used. Examples of suitable materials include plastics and other materials used in the manufacture of conventional catheters, and newly-developed materials determined to be suitable for medical catheters. The shaft 20 may also be made from appropriate material or include an appropriate configuration and/or arrangement of elements and/or parts to confer and/or enhance the torquability of the shaft 20 and/or the entire catheter 10. For example, the shaft 20 can be reinforced with a braiding and/or a cannula. If used, these structures can be attached to a surface of the shaft 20, such as at one or more bonding points on the surface of the shaft 20, and/or embedded within the material of the shaft 20, as is known in the art.

Those skilled in the art recognize that there are many ways in which a catheter assembly may be introduced into a bodily passage, and accordingly, the catheter assembly may be introduced in any of these well-known ways. These well-known ways including, but not limited to inserting a catheter assembly into a bodily passage, introducing a catheter assembly 10 over a wireguide that has previously been inserted into a bodily passage (as illustrated in FIGS. 1 through 4), passing the catheter assembly through a sheath introduced into the bodily passage (discussed below), and utilization of a rapid exchange style configuration.

The catheter 10 defines a wireguide lumen 13 that extends through the shaft 20, as best illustrated in FIG. 3. The wireguide lumen 13 extends from the proximal end 27 of the catheter 10 to the distal end 28 of the catheter 10. The catheter 10 illustrated in FIGS. 1 through 4 is a dual lumen catheter, including the wireguide lumen 13 and the inflation lumen 18 (discussed above). While the illustrated catheter 10 includes two lumens, catheters according to particular embodiments can include any desired number of lumens, including one, two, three, four or more lumens. Skilled artisans will be able to determine the appropriate number of lumens based upon various considerations, e.g., intended use. The catheter 10 can be free of attachment to the wireguide 12, as illustrated in the Figures, or, alternatively, the wireguide 12 can be attached to a portion of the catheter, such as an interior portion adjacent the wireguide lumen 13, such that the catheter 10 and wireguide 12 can be advanced and retracted as a single unit.

A balloon 40 is attached to the distal portion 24 of the shaft 20 at a proximal junction 60 and a distal junction 61. Junctions 60, 61 can comprise any suitable attachment between members, and skilled artisans will be able to select an appropriate attachment for a particular catheter based on various considerations, including the nature of the materials used. Examples of suitable attachments include attachments formed by heat fusion techniques and/or procedures, adhesive attachments, mechanical connections, and any other suitable attachment between members. The inventor has determined that an attachment formed by heat fusing the distal balloon neck 46 to the shaft 20 and by heat fusing the proximal balloon neck 44 to the shaft 20 is suitable. Adhesives, connectors, and other suitable structure and/or compositions can be used to form a suitable junction 60, 61. No matter the type or form of attachment used, the junctions 60, 61 should sufficiently seal the proximal balloon neck 44 and the distal balloon neck 46 to the shaft 20 so that fluid within the inflation lumen 18 cannot pass through the junction 60, 61 during inflation of the balloon 40, described more fully below.

The material of the balloon 40 and the portion of the exterior surface of the shaft 20 positioned within the balloon 40 define an inflation chamber 43. The balloon 40 is positioned on the distal portion 24 of the shaft 20 such that the opening 41 is fluidly connected to the inflation chamber 43. With this structural arrangement, the balloon 40 is adapted to move between deflated and inflated configurations as a fluid is moved into and out of the inflation chamber 43 via the inflation lumen 18 and the opening 41.

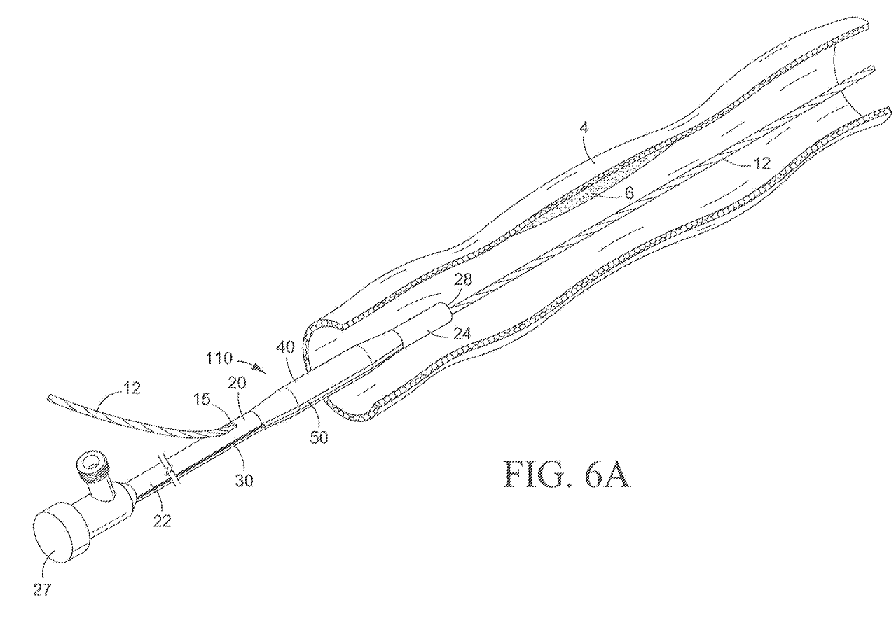

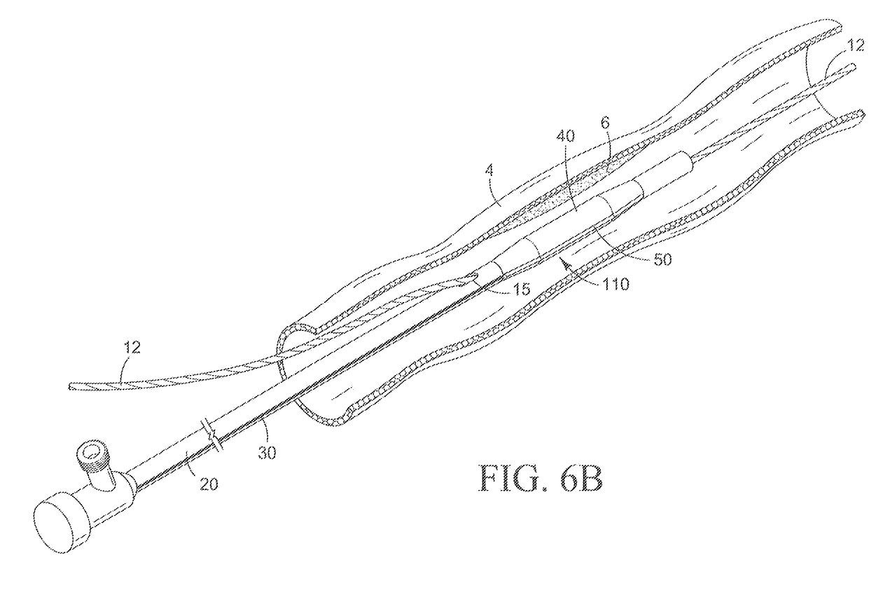

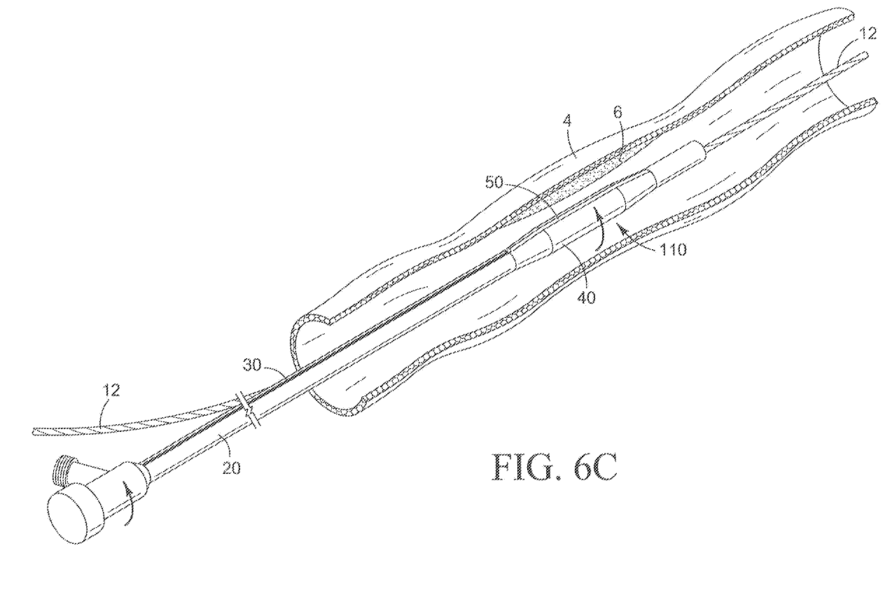

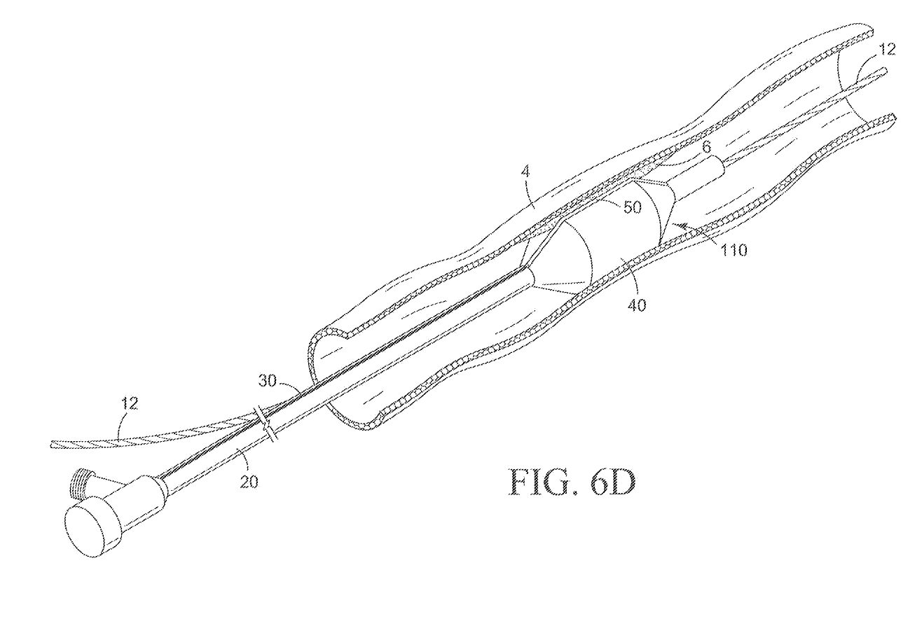

A care provider inflates the balloon 40 by introducing an appropriate fluid, such as saline, into the inflation lumen 18 until the fluid passes through opening 41 and into the inflation chamber 43. The resulting pressure placed on the inner surface of the balloon 40 by the fluid causes the balloon 40 to inflate and adopt the inflated configuration. To move the balloon to the deflated configuration, vacuum pressure can be applied to the inflation lumen 18 to remove fluid located within the inflation chamber 43 via the opening 41, resulting in the balloon 40 collapsing against the distal portion 24 of the shaft 20. FIG. 1 through FIG. 5, as well as FIG. 6D, illustrate the balloon 40 in the inflated configuration. FIGS. 6A through 6C illustrate a second exemplary balloon 40 in the deflated configuration.

Additional structure can be attached to the catheter 10 to facilitate the inflation and deflation of the balloon 40 as described above. For example, a syringe (not illustrated) or other suitable structure can be attached to the inflation port 16 using any suitable connection, such as a Luer lock connector. The fluid can be stored within the syringe and inflation lumen 18, and can be introduced into and removed from the inflation chamber 43 by operating the syringe using conventional practices.

The balloon 40 has an axial length extending along the longitudinal axis of the shaft 20 and has a proximal balloon neck 44 and a distal balloon neck 46. An intermediate portion 49 is disposed between the proximal balloon neck 44 and distal balloon neck 46. The intermediate portion 49 extends from an intermediate portion proximal end 54 to an intermediate portion distal end 56.

As best illustrated in FIG. 2, the intermediate portion 49 defines a maximum diameter 58 of the balloon 40 when the balloon 40 is in the inflated configuration. The maximum diameter 58 is uniform along the axial length of the intermediate portion 49 of the balloon 40. In contrast, the proximal balloon neck 44 and distal balloon neck 46 of the balloon 40 have diameters that taper along the axial length of the respective balloon necks 44, 46; tapering from a minimal diameter positioned at the locations where the balloon necks 44, 46 contact the shaft 20 to the maximum diameter 58 at the locations where the balloon necks 44, 46 meet the intermediate portion 49.

As best illustrated in FIG. 2, the balloon 40 has a working length 52 that extends along the lengthwise axis of the shaft 20 from the intermediate portion proximal end 54 to the intermediate portion distal end 56. The working length 52 is an axial portion of the balloon 40 that has the maximum diameter 58 of the balloon 40 when the balloon 40 is in the inflated configuration. Thus, in use, the working length 52 of the balloon 40 corresponds to the portion of the balloon 40 extending along the axial length of the shaft 20 that contacts an inner wall or tissue of a bodily passage when the balloon 40 is in the inflated configuration while disposed within the bodily passage.

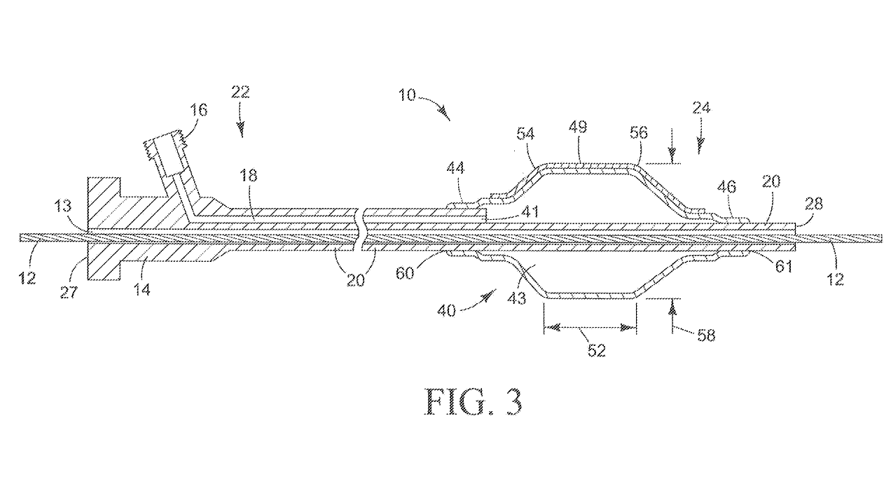

The balloon 40 has an outer surface 42. The balloon 40 outer surface 42 defines a balloon arc 48. As best illustrated in FIG. 5, the balloon arc 48 extends between a proximal endpoint 45 on the proximal balloon neck 44, and a distal endpoint 47 on the distal balloon neck 46. The proximal endpoint 45 is located near the junction 60, whereas the distal endpoint 47 is located near the junction 61.

It is advantageous for balloon 40 to be a non-compliant balloon of the type commonly used with a balloon catheter. Such balloons are well known in the art, and are typically extruded from polymeric materials such as various polyamides (e.g., nylons), polyethylene terephthalate (PET), polyether block amides (PEBA), and mixtures of the foregoing. Non-compliant balloons are well known for their ability to maintain their inflated diameter under high-pressure conditions of the type that may be encountered during use of a balloon catheter. Although non-compliant balloons are considered desirable for at least this reason, those skilled in the art will appreciate that compliant balloons may be acceptable in some circumstances, such as when less severe conditions are expected to be encountered. Catheter balloons are well known, and those skilled in the art can readily select an appropriate balloon for use in carrying out a particular procedure.

As illustrated in FIGS. 1 through 5, balloon 40 is provided with at least one raised element 50 distributed along the outer surface 42 of the balloon 40. The raised element 50 comprises one or more raised elements. The raised element 50 is arranged along the outer surface 42 of balloon 40 in a manner that that enables the raised element 50 to dilate an airway stenosis. The raised element is disposed axially along at least a portion of the balloon arc 48, but can also extend along the entire balloon arc 48, such as from the proximal endpoint 45 to the distal endpoint 47. In another exemplary catheter, the raised element 50 is disposed axially along at least a portion of the balloon arc 48 between the intermediate portion proximal end 54 and the intermediate portion distal end 56.

The raised element 50 can be made from any suitable material, and need only be able to perform as described herein. Skilled artisans will be able to select an appropriate material for a raised element or elements in a catheter according to a particular embodiment based on various considerations, including the type of bodily passages within which the catheter is intended to be used. Examples of suitable materials include polymeric materials and metals. Polymeric materials are considered advantageous at least because of the ease of handling and well-characterized nature. Forming the raised element from the same base polymer that is used to form the balloon is considered particularly advantageous at least because it is expected to provide efficiencies in the manufacture of catheters. It is noted, though, that the raised element can be formed of a base polymer that is unrelated to the base polymer of the balloon. The raised element 50 should have sufficient strength and rigidity to dilate airway stenosis encountered upon the introduction of the balloon 40 into a bodily passage, as discussed hereinafter.

As stated, the raised element 50 and the balloon 40 can be formed from the same or a similar polymer. When the same or similar polymers are utilized for the balloon and the raised element, the bonding of such polymers will normally result in a very favorable and strong seal there between. In addition, when the raised element is formed of the same or a similar polymer as the balloon, the raised element has the ability to expand as the balloon is expanded, e.g., during inflation of the balloon.

Although it is considered advantageous to utilize the same or a similar polymer for the raised element and the balloon, it is not necessary to do so. Rather, any compositions for the respective raised element and balloon that are suitable for the intended purpose may be substituted, as long as the respective compositions are sufficiently compatible such that the raised element may be securely bonded or otherwise attached to the outer circumference of the balloon. Furthermore, the raised element need not even be formed from a polymer, and other materials, such as metals, alloys (including shape memory alloys), composites, etc., may be appropriate for a particular usage.

The raised element 50 can be integrally formed with the balloon 40. For example, the raised element can comprise an axial portion of the balloon having a greater thickness than adjacent axial portions of the balloon. Also, the raised element can comprise an axial portion of the balloon that defines an outwardly-facing edge.

Alternatively, the raised element 50 can comprise a separate element that is attached to balloon outer surface 42 by any well-known method utilized in the medical arts for joining components. This structural arrangement and relationship is considered particularly advantageous for raised elements formed of metal. In these embodiments, the raised element can be bonded to the outer surface 42 of balloon 40 using any suitable bonding techniques, methods, procedures, and/or agents. Heat bonding is a suitable technique that may be used for forming a strong bond between polymeric compositions. Heat bonding is a well-known technique, and those skilled in the art can readily determine appropriate conditions upon which a particular bonding operation may be carried out. Laser-welding of the raised element to the outer surface of the balloon is also considered suitable. Other techniques suitable for a particular bonding operation include, but are not limited to, adhesive bonding and/or solvent bonding. When bonding techniques such as adhesive and/or solvent bonding are carried out, those skilled in the art will appreciate that only biologically compatible materials should be used. Other known attachment means may be used in place of, or in combination with, bonding. As another alternative, the raised element may be bonded or otherwise attached to the balloon during the process of forming the balloon. As still another alternative, balloon 40 can be formed with an integral raised element 50.

In the illustrated embodiment, the raised element 50 is a single elongated raised element that spans an axial length of the balloon 40. It is noted that the raised element need not span the entire axial length of balloon 40, and can merely span a portion of the length. Indeed, the raised element 50 can span any suitable axial length of the balloon 40, including an axial length equal to about the entire axial length, an axial length equal to between about 10% and about 90% of the entire axial length, an axial length equal to between about 25% and about 75% of the entire axial length, an axial length equal to between about 45% and about 55% of the entire axial length, and an axial length equal to about 50% of the entire axial length. Furthermore, a plurality of discrete raised elements may be included instead of a single elongated raised element. The configuration illustrated in the drawings and described herein is exemplary only, and those skilled in the art will appreciate that more rows of raised elements may be provided. Similarly, each row need not include a single raised element as illustrated in the figures, and may include more raised elements.

As illustrated in FIGS. 1 through 3, the catheter assembly 10 comprises an elongate shaft 20. The elongate shaft 20 has a proximal portion 22 and a distal portion 24. The proximal portion 22 and the distal portion 24 define an axial length there-between. The elongate shaft 20 has an exterior surface 26. The exterior surface 26 defines a marking indicia 30 that extends axially along at least a portion of the elongate shaft 20. In the illustrated embodiment, the marking indicia 30 extends along the entire axial length from junction 60 to proximal portion 22 of the shaft 20.

Some balloon catheters of the type traditionally used in vasculature have one or more radiopaque marking indicia located on the shaft of the balloon catheter. Radiopaque marking indicia are used because x-ray fluoroscopy or other imaging techniques are needed to indirectly confirm the position of the balloon in the vasculature as well as the working length of the balloon. To facilitate the care provider in determining the location and orientation of the balloon on these catheters, the radiopaque marking indicia are typically located inside the inflation chamber defined by the balloon. For catheters intended to be used with direct visualization techniques, such as direct visualization with a scope, marking indicia disposed within the inflation chamber would be disadvantageous because the user may not be able to see the marking indicia under visualization.

As illustrated in FIGS. 1 and 2, the elongate shaft 20 comprises at least one marking indicia 30 located on the exterior surface 26 of the shaft 20. The marking indicia 30 illustrated in those Figures comprises one or more lines or stripes. The marking indicia 30 illustrated in FIGS. 1 and 2 extends along the shaft 20. In other exemplary catheters, the marking indicia could extend onto the fitting 14; could extend onto the cutting balloon 40, for instance the marking indicia could extend onto the proximal balloon neck 44 and/or the distal balloon neck 46; or could extend onto other attachments to the catheter assembly. The marking indicia could also be non-linear. One exemplary non-linear marking indicia comprising a single indicator, such as a round dot.

The marking indicia 30 can be formed in any suitable manner. For instance, as illustrated in FIGS. 1 and 2, the marking indicia 30 is a stripe printed on, painted onto, or attached to the outside surface of the shaft 20. In another exemplary catheter, the marking indicia is embedded into the exterior surface of the shaft with a translucent portion of the shaft allowing the marking indicia to be viewed therethrough. In another exemplary embodiment, the marking indicia can comprise a material embedded into the shaft. For example, the marking indicia can be a contrasting colored polymer portion embedded into the shaft. Alternatively, the marking indicia can comprise a different material than the shaft. In another exemplary catheter, the marking indicia is an engraved mark or marks on the shaft. While FIGS. 1 and 2 disclose a single line comprising the marking indicia 30, those skilled in the art can readily select the necessary marking indicia, or combinations of marking indicia, necessary for use in carrying out a particular procedure, e.g., multiple lines, varying thicknesses of lines, continuous lines, interrupted lines, non-linear indicia, colors, patterns, depressions, protrusions, protrusions on the proximal and/or distal balloon necks.

During use of the catheter assembly 10, the balloon 40 is initially in its deinflated configuration. In this configuration, the edge of the raised element 50, which can be a cutting edge, is shielded or otherwise covered by the folds defined by the deflated balloon 40. As a result, the cutting edge is not exposed to the portions of the bodily passage that are traversed during the introduction of catheter assembly 10 into the bodily passage. In addition, this arrangement protects medical personnel by reducing the possibility of inadvertent contact with a sharp cutting edge. Depending upon how the balloon is folded, however, such shielding/covering may not be necessary.

A sheath may also be employed for protecting the cutting edge of the raised element 50 from contacting the portions of the bodily passage that are traversed during the introduction of the catheter assembly into the bodily passage.

A sheath might also enable the catheter 10 to be rotated easier. Further, the sheath and balloon might be rotated together.

After visually verifying that the marking indicia is aligned as desired with the target lesion, a user can inflate the balloon 40 using conventional techniques, as discussed herein. Inflation of the balloon 40 causes the cutting edge of the raised element 50 to engage the tissue of the lesion. Contact between the raised element 50 and the tissue while the balloon 40 is inflated can form disruptions in the surface of the tissue in the lesion, such as cuts, scores and/or other disruptions. The balloon may be deflated and re-inflated one or more additional times, if necessary and/or desired, to achieve the formation of a suitable or desired number and/or quality of disruptions in the tissue. The assembly may also be rotated prior to re-inflation such that additional segments or portions of the lesion may be directly engaged by the raised element 50.

Following formation of disruptions in the surface of the tissue in the lesion, the balloon 40 is deflated such that it returns to the deflated configuration. The balloon catheter assembly may then be retracted over the wireguide and removed from the bodily passage. Alternatively, the balloon catheter assembly and wireguide can be removed together if, for example, the wireguide is fixed to a portion of the balloon catheter assembly, as described above.

In the exemplary catheter of FIGS. 1 and 2, the marking indicia 30 extends generally parallel to the axial length of the shaft, from the proximal portion 22 to the distal portion 24. The marking indicia 30 may extend the entire axial length of the shaft, or a portion thereof. The exemplary catheter 10 of FIGS. 1 and 2 illustrating a first marking indicia 30 on the proximal side of the balloon 40, and a second marking indicia 130 on the distal side of the balloon 40. In some exemplary embodiments, the second marking indicia 130 may not be present. The first and second marking indicia 30, 130 are illustrated as being axially aligned with each other in FIGS. 1 and 2, however in other exemplary catheters the first and second marking indicia 30, 130 may not be axially aligned with each other. While in the exemplary catheter illustrated in FIGS. 1 and 2, the marking indicia 30 extends generally parallel to the axial length of the shaft, the marking indicia may be non-linear, for instance comprising a single indicator, such as a round dot.

In the exemplary catheter 10 of FIGS. 1 and 2, the raised element 50 is illustrated as being aligned with both of the marking indicia 30, 130. Thus, raised element 50 is axially aligned with the lengthwise axis of elongate shaft 20 and marking indicia 30, 130. Each marking indicia 30, 130 is adapted to indicate the location of the raised element 50 about the circumference of the elongate shaft 20. In such a configuration, proximal endpoint 45 is located in-line with the first marking indicia 30, and the distal endpoint 47 is located in-line with the second marking indicia 130. Again, in some exemplary embodiments, the second marking indicia would not be present. This allows a care provider to directly view a portion of the shaft 20, and, by viewing the location and orientation of the marking indicia, thereby confirm the alignment of the raised element 50 attached thereto.

In another exemplary catheter, a plurality of raised elements are provided, and a plurality of marking indicia are provided on the shaft. In such an exemplary catheter, each marking indicia could correspond to and/or be aligned with a different raised element.

While raised element 50 has been illustrated and described as being axially aligned with both marking indicia 30, 130 and the lengthwise axis of elongate shaft 20, a raised element can be aligned in any suitable manner with respect to marking indicia on an elongate shaft. Skilled artisans will be able to select a suitable orientation to align a raised element with respect to a marking indicia according to a particular embodiment based on various considerations, including the location of a desired point of treatment. Example orientations considered suitable to align a raised element with respect to a marking indicia include, but are not limited to, orienting a raised element such that it is axially aligned with a marking indicia, orienting a raised element such that it is axially aligned with a marking indicia and the lengthwise axis of an elongate shaft, orienting a raised element such that it is not axially aligned with a marking indicia, and orienting a raised element such that it is circumferentially aligned with a marking indicia. For example, a marking indicia can be disposed opposite, or substantially opposite, a raised element about the circumference of an elongate shaft such that it is axially aligned with a raised element and the lengthwise axis of an elongate shaft.

A raised element can be circumferentially aligned with a marking indicia in any suitable manner. When a raised element is circumferentially aligned with a marking indicia, an axis of raised element is disposed on a first plane and an axis of the marking indicia is disposed on a second plane that intersects the first plane at a defined angle. Thus, a raised element can be disposed at any suitable location about the circumference of an elongate shaft with respect to marking indicia. Skilled artisans will be able to select a suitable location to dispose a raised element about the circumference of an elongate shaft with respect to a marking indicia according to a particular embodiment based on various considerations, including the location of a desired point of treatment. For example, a raised element can be circumferentially aligned with a marking indicia at any angle between about 0 degrees to about 360 degrees. Other angles considered suitable to circumferentially align a raised element with a marking indicia include, but are not limited to, angles between about 1 degree to about 180 degrees, and angles between about 180 degrees to about 360 degrees. Additional angles considered suitable to circumferentially align a raised element with a marking indicia include, but are not limited to, an orthogonal angle, a substantially orthogonal angle, a 45 degree angle, a substantially 45 degree angle, a 135 degree angle, a substantially 135 degree angle, and any other angle considered suitable for a particular application. Optionally, each of a first plane containing an axis of a raised element and a second plane containing an axis of a marking indicia can contain the lengthwise axis of an elongate shaft and intersect at a defined angle, such as those described herein.

Orienting a raised element such that it is not axially aligned with a marking indicia and the lengthwise axis of an elongate shaft is considered advantageous at least because this configuration provides a care provider with an indication as to where a raised element does not exist on a balloon. In use, this is considered advantageous at least because it provides a mechanism for orienting a marking indicia with healthy tissue within a bodily passage and positioning the raised element away from the healthy tissue. Furthermore, orienting a raised element such that it is not axially aligned with a marking indicia about the circumference of an elongate shaft is considered advantageous at least because it conveys information relating to where a raised element is positioned on a balloon during use. For example, marking indicia can be positioned opposite, or substantially opposite, a raised element about the circumference of an elongate shaft or at an orthogonal, or substantially orthogonal, angle to a raised element.

FIGS. 6A through 6D illustrate a second exemplary catheter 110. The catheter 110 illustrated in FIGS. 6A through 6D is similar to the first exemplary catheter 10 illustrated in FIGS. 1 through 5, except as detailed below. Thus, the catheter 110 includes a catheter shaft 20, marking indicia 30, balloon 40, and raised element 50. The second exemplary catheter 110 having a first marking indicia 30, but, unlike the first exemplary catheter 10 of FIGS. 1 through 5, a second marking indicia 130 is not provided.

FIGS. 6A through 6D illustrate, sequentially, the second exemplary catheter 110 in various configurations adopted during use. FIGS. 6A through 6D show the second exemplary catheter 110 used to perform dilation of a stenosis 6 in a bodily passage 4.

Those skilled in the art recognize that there are many ways in which a catheter assembly may be introduced into a bodily passage, and accordingly, the catheter assembly may be introduced in any of these well-known ways. For instance, the catheter assembly 110 can be introduced over a wireguide that has previously been inserted into a vessel, such as is illustrated in FIGS. 6A through 6D.

FIGS. 6A through 6D illustrate a “rapid exchange” style catheter where the catheter 110 has an exchange port 15 defined in the shaft 20. The exchange port 15 extends through the shaft 20 from the outside surface of the shaft to a wireguide lumen. It is considered desirable for the exchange port 15 to be located through the shaft 20 in a location not interrupting or otherwise interfering with visualization of the marking indicia 30. The wireguide lumen is illustrated extending through the shaft 20 to the distal end 28 of the shaft 20. The wireguide 12 is illustrated extending through the wireguide lumen of the shaft 20 and through a bodily passage 4. It is noted that, while rapid exchange configurations provide certain advantages, a disadvantage to catheters configured in this manner is that the wireguide can wrap around the shaft when the shaft is rotated, which may make withdrawal of the catheter more difficult.

Referring to FIG. 6A, a distal portion, such as the distal end 28 of a balloon catheter assembly 110 is inserted into a bodily passage 4 and over a previously placed wireguide 12. The balloon catheter assembly 110 is then advanced along the wireguide 12. Alternatively, as described above, the balloon catheter assembly 110 and wireguide 12 can be inserted into the bodily passage 4 and advanced together if the wireguide 12 is attached to a portion of the balloon catheter assembly 110.

The balloon catheter assembly 110 comprises an elongate catheter shaft 20 and an inflatable balloon 40. The elongate catheter shaft 20 comprises a proximal portion 22 and a distal portion 24. The elongate catheter shaft 20 defines an axial length between the proximal portion 22 and the distal portion 24. The elongate catheter shaft 20 defines marking indicia 30 along at least a portion of the axial length. The marking indicia 30 is oriented generally parallel to the axial length. The inflatable balloon 40 has a raised element 50 aligned with the marking indicia 30. Thus, raised element 50 is axially aligned with the lengthwise axis of elongate shaft 20 and marking indicia 30. The marking indicia 30 is adapted to indicate the location of the raised element 50 about the circumference of the elongate shaft 20.

Referring now to FIG. 6B, the balloon catheter assembly 110 is then advanced into the bodily passage 4 with the balloon 40 in an uninflated condition (also illustrated in FIGS. 6A and 6C) until the balloon 40 reaches the stenosis. The care provider then observes, for instance through the use of an endoscope or other direct visualization technique or equipment, the orientation of the marking indicia 30 on the shaft 20 relative to the bodily passage 4 and/or the lesion 6 in the bodily passage 4. Observation of the marking indicia 30 allows the care provider to confirm the orientation of the raised element 50 relative to the lesion.

Referring now to FIG. 6C, the care provider then adjusts the positioning of the catheter shaft 20 in the bodily passage 4 to align the raised element 50 with the lesion 6. For instance, rotation of the catheter shaft 20, or rotation of both the sheath and balloon shaft if the balloon is advanced through a sheath, can be accomplished in order to the position the catheter shaft 20 as desired by the care provider. Additional observation, direct visualization and positioning may be necessary before the care provider is confident that the raised element 50 is appropriately aligned.

Referring now to FIG. 6D, the care provider would then inflate the balloon 40 such that the raised element 50 contacts the surface of the tissue in the lesion 6. Contact between the raised element 50 and the tissue while the balloon 40 is inflated can form disruptions in the surface of the tissue in the lesion, such as cuts, scores and/or other disruptions. The balloon may be deflated and re-inflated one or more additional times, if necessary and/or desired, to achieve the formation of a suitable or desired number and/or quality of disruptions in the tissue. The catheter 10 may also be rotated prior to re-inflation such that additional segments or portions of the lesion may be directly engaged by the raised element 50.

Upon completion of the procedure, the balloon 40 would be deflated and the balloon catheter assembly 110 would be withdrawn from the bodily passage 4 over the wireguide 12. Alternatively, as described above, the balloon catheter assembly 110 and wireguide 12 can be withdrawn from the bodily passage 4 together if the wireguide is attached to a portion of the balloon catheter assembly 110.

While FIGS. 6A through 6D illustrate the advancement of a balloon catheter assembly 110 over a previously placed wireguide 12, a wireguide does not have to be used for this method.

Thus, in using the catheter 110, a care provider is able to verify the placement of the catheter 110 within a bodily passage by directly viewing (e.g., via an endoscope) the marking indicia 30 on a portion of the shaft 20 of the catheter 110. Based upon the observed orientation marking indicia 30 relative to the bodily passage, the care provider can rotate the shaft 20 of the catheter 110 to bring raised elements 50 on a balloon 40 into alignment with a lesion to be dilated.

FIG. 7 is a flowchart representation of a method 200 of treating a portion of a bodily passage. An initial step 201 comprises inserting a catheter into a bodily passage. The catheter comprises an elongate shaft having a proximal end and a distal end. The catheter further defines an inflation lumen. A balloon is attached to the distal end of the shaft and is adapted to move between deflated and inflated configurations. The balloon has a proximal portion, a distal portion, and an intermediate portion disposed between the proximal and distal portions. The intermediate portion of the balloon defines a maximum diameter of the balloon when the balloon is in the inflated configuration. The balloon is in fluid communication with the inflation lumen via an opening defined by the main body of the catheter. A marking indicia is disposed on or in the shaft, and is aligned (e.g., axially aligned, circumferentially aligned) with one or more raised elements formed by or disposed on the balloon.

Another step 202 comprises advancing the catheter through the bodily passage with the balloon in an uninflated condition until the uninflated balloon reaches the intended point of treatment, such as a stenosis or other lesion within the passage.

Following the step of advancing the catheter 202, another step 203 comprises confirming the orientation of the catheter relative to the portion of the bodily passage intended to be treated, such as the stenosis or other lesion. This confirmation can be performed using direct visualization equipment, such as a scope, and techniques or other suitable equipment and techniques. Advantageously, this step is performed using the selected visualization equipment and technique to visualize a portion of the shaft and the location of the marking indicia on the shaft relative to the portion of the bodily passage intended to be treated, such as the stenosis or other lesion.

Another step 204 comprises positioning the catheter to align the one or more raised elements with the portion of the bodily passage intended to be treated by the catheter. Positioning the catheter may include rotation of the catheter and/or laterally adjusting the catheter to align the portion of the bodily passage intended to be treated by the catheter with the catheter.

An optional step 205 comprises confirming the orientation of the catheter and/or the one or more raised elements relative to the portion of the bodily passage intended to be treated by the catheter following performance of step 204. This step 205 can be included to confirm that the catheter is correctly aligned. If the catheter is not correctly aligned, the catheter may be repositioned as necessary by repeating step 204 and, optionally, again repeating this step 205.

Another step 206 comprises inflating the balloon of the catheter. This step can be used to perform dilation of the portion of the bodily passage intended to be treated by the catheter, such as the stenosis or other lesion. For example, this step can be used to perform dilation of an airway stenosis through use of a cutting balloon. This step 206 can be performed using conventional techniques, as discussed herein. Inflation of the balloon causes the cutting edge of the raised element on the balloon to engage the tissue of the lesion. Contact between the raised element and the tissue while the balloon is inflated can form disruptions in the surface of the tissue in the lesion, such as cuts, scores and/or other disruptions.

Following the step 206 of inflating the balloon, a step 207 of deflating the balloon is performed.

To allow repeat dilations and/or the formation of multiple and/or deeper disruptions in the surface of the tissue intended to be treated, one or more of the previous steps 204 through step 207 may be repeated, if necessary and/or desirable. For example, the steps of inflating 206 and deflating 207 the balloon can be successively repeated to achieve a desired number of repetitions, disruptions, and/or quality of disruptions in the surface of the tissue.

Another step 208 comprises withdrawing the catheter from the bodily passage.

FIG. 8 is a flowchart representation of a method 300 of treating a portion of a bodily passage. An initial step 301 comprises inserting a sheath into a bodily passage of a patient using conventional interventional techniques.

Another step 302 comprises inserting a catheter into the sheath. The catheter comprises an elongate shaft having a proximal end and a distal end. The catheter further defines an inflation lumen. A balloon is attached to the distal end of the shaft and is adapted to move between deflated and inflated configurations. The balloon has a proximal portion, a distal portion, and an intermediate portion disposed between the proximal and distal portions. The intermediate portion of the balloon defines a maximum diameter of the balloon when the balloon is in the inflated configuration. The balloon is in fluid communication with the inflation lumen via an opening defined by the main body of the catheter. A marking indicia is disposed on or in the shaft, and is aligned (e.g., axially aligned, circumferentially aligned) with one or more raised elements formed by or disposed on the balloon.

Another step 303 comprises advancing the catheter through the sheath, and thus through the bodily passage, with the balloon in an uninflated condition until the uninflated balloon exits the sheath and is disposed adjacent the intended point of treatment, such as a stenosis or other lesion within the passage.

Following the step of advancing the catheter 303, another step 304 comprises confirming the orientation of the catheter relative to the portion of the bodily passage intended to be treated, such as the stenosis or other lesion. This confirmation can be performed using direct visualization equipment, such as a scope, and techniques or other suitable equipment and techniques. Advantageously, this step is performed using the selected visualization equipment and technique to visualize a portion of the shaft and the location of the marking indicia on the shaft relative to the portion of the bodily passage intended to be treated, such as the stenosis or other lesion.

Another step 305 comprises positioning the catheter to align the one or more raised elements with the portion of the bodily passage intended to be treated by the catheter. Positioning the catheter may include rotation of the catheter and/or laterally adjusting the catheter to align the portion of the bodily passage intended to be treated by the catheter with the catheter.

An optional step 306 comprises confirming the orientation of the catheter and/or the one or more raised elements relative to the portion of the bodily passage intended to be treated by the catheter following performance of step 305. This step 306 can be included to confirm that the catheter is correctly aligned. If the catheter is not correctly aligned, the catheter may be repositioned as necessary by repeating step 305 and, optionally, again repeating this step 306.

Another step 307 comprises inflating the balloon of the catheter. This step can be used to perform dilation of the portion of the bodily passage intended to be treated by the catheter, such as the stenosis or other lesion. For example, this step can be used to perform dilation of an airway stenosis through use of a cutting balloon. This step 307 can be performed using conventional techniques, as discussed herein. Inflation of the balloon causes the cutting edge of the raised element on the balloon to engage the tissue of the lesion. Contact between the raised element and the tissue while the balloon is inflated can form disruptions in the surface of the tissue in the lesion, such as cuts, scores and/or other disruptions.

Following the step 307 of inflating the balloon, a step 308 of deflating the balloon is performed.

To allow repeat dilations and/or the formation of multiple and/or deeper disruptions in the surface of the tissue intended to be treated, one or more of the previous steps 305 through step 308 may be repeated, if necessary and/or desirable. For example, the steps of inflating 307 and deflating 308 the balloon can be successively repeated to achieve a desired number of repetitions, disruptions, and/or quality of disruptions.

Another step 309 comprises withdrawing the catheter from the sheath, and thus from the bodily passage.

Another step 310 comprises withdrawing the sheath from the bodily passage.

It is noted that the inventive methods can be used in any suitable bodily passage in any suitable animal. For example, the methods can be used in the treatment of human beings and other animals, and can be used in the treatment of sinus cavities, airway passages, and any other bodily passage for which treatment is desired. While the use of a catheter shaft having marking indicia is considered particularly advantageous for inclusion in methods used in pulmonary airways, the inventor does not consider the apparatuses or methods limited to use in such bodily passages.

The foregoing detailed description provides exemplary embodiments of the invention and includes the best mode for practicing the invention. The description and illustration of these embodiments is intended only to provide examples of the invention, and not to limit the scope of the invention, or its protection, in any manner.

What is claimed is:

1. A balloon catheter comprising:
an elongate shaft having a proximal end, a distal end, a lengthwise axis, an exterior surface, a circumference, and an axial length extending along the lengthwise axis from the proximal end to the distal end, the elongate shaft defining an inflation lumen;
a marking indicia disposed on the elongate shaft and extending axially along at least a portion of the axial length of the elongate shaft;
an inflatable balloon attached to the distal end of the elongate shaft and cooperatively defining an inflation chamber with a portion of the exterior surface of the elongate shaft, the inflation chamber in fluid communication with the inflation lumen, the inflatable balloon having deflated and inflated configurations and adapted to move from the deflated configuration to the inflated configuration as fluid moves into the inflation chamber from the inflation lumen; and
a raised element disposed on the inflatable balloon and aligned with the marking indicia;
wherein the marking indicia is adapted to indicate the location of the raised element about the circumference of the elongate shaft.

2. The balloon catheter of claim 1, wherein the raised element is axially aligned with the lengthwise axis of the elongate shaft and the marking indicia.

3. The balloon catheter of claim 1, wherein the raised element is circumferentially aligned with the marking indicia.

4. The balloon catheter of claim 1, wherein the marking indicia is embedded within the elongate shaft.

5. The balloon catheter of claim 1, wherein the inflatable balloon has an outer surface, a proximal end and a distal end;
wherein the outer surface defines a balloon arc between a proximal endpoint on said proximal end and a distal endpoint on said distal end; and
wherein at least one of the proximal and distal endpoints is disposed adjacent the marking indicia.

6. The balloon catheter assembly of claim 5, wherein the proximal endpoint is adjacent the marking indicia.

7. The balloon catheter assembly of claim 5, wherein the distal endpoint is adjacent the marking indicia.

8. The balloon catheter of claim 1, wherein the raised element is integrally formed with the inflatable balloon.

9. The balloon catheter of claim 1, wherein the inflatable balloon has an outer surface and the raised element comprises a separate element attached to the outer surface.

10. The balloon catheter of claim 9, wherein the raised element is formed of metal.

11. The balloon catheter of claim 10, wherein the raised element defines a cutting edge.

12. The balloon catheter of claim 11, wherein the inflatable balloon is configured to cover the cutting edge of the raised element when the balloon is in the deflated configuration.

13. The balloon catheter of claim 1, wherein the inflatable balloon has an axial balloon length; and
wherein the raised element extends axially along only a portion of the axial balloon length.

14. The balloon catheter of claim 1, wherein the inflatable balloon has an axial balloon length; and
wherein the raised element extends axially along the entire axial balloon length.

15. A balloon catheter comprising:
an elongate shaft having a proximal end, a distal end, a lengthwise axis, an exterior surface, a circumference, and an axial length extending along the lengthwise axis from the proximal end to the distal end, the elongate shaft defining an inflation lumen;
a marking indicia disposed on the elongate shaft and extending axially along at least a portion of the axial length of the elongate shaft;
an inflatable balloon attached to the distal end of the elongate shaft and cooperatively defining an inflation chamber with a portion of the exterior surface of the elongate shaft, the inflatable balloon having an outer surface, a proximal end, a distal end, and an axial length, the outer surface defining a balloon arc extending between a proximal end point on the proximal end of the balloon and a distal endpoint on the distal end of the balloon, the inflation chamber in fluid communication with the inflation lumen, the inflatable balloon having deflated and inflated configurations and adapted to move from the deflated configuration to the inflated configuration as fluid moves into the inflation chamber from the inflation lumen; and
a raised element disposed on the inflatable balloon and aligned with the marking indicia;
wherein at least one of the proximal endpoint and the distal endpoint is disposed adjacent the marking indicia; and
wherein the marking indicia is adapted to indicate the location of the raised element about the circumference of the elongate shaft.

16. The balloon catheter of claim 15, wherein the raised element is axially aligned with the lengthwise axis of the elongate shaft and the marking indicia.

17. The balloon catheter of claim 15, wherein the raised element is circumferentially aligned with the marking indicia.

18. The balloon catheter of claim 15, wherein the raised element is integrally formed with the inflatable balloon.

19. The balloon catheter of claim 15, wherein the raised element comprises a separate element attached to the outer surface.

20. A method of treating a portion of a bodily passage, said method comprising:
inserting a catheter into said bodily passage, the catheter comprising:
an elongate shaft having a proximal end, a distal end, a lengthwise axis, an exterior surface, a circumference, and an axial length extending along the lengthwise axis from the proximal end to the distal end, the elongate shaft defining an inflation lumen;
a marking indicia disposed on the elongate shaft and extending axially along at least a portion of the axial length of the elongate shaft;
an inflatable balloon attached to the distal end of the elongate shaft and cooperatively defining an inflation chamber with a portion of the exterior surface of the elongate shaft, the inflation chamber in fluid communication with the inflation lumen, the inflatable balloon having deflated and inflated configurations and adapted to move from the deflated configuration to the inflated configuration as fluid moves into the inflation chamber from the inflation lumen; and
a raised element disposed on the inflatable balloon and aligned with the marking indicia;
advancing the catheter through the bodily passage with the balloon in an uninflated configuration until the uninflated balloon reaches an intended point of treatment within said bodily passage;
following the step of advancing the catheter, confirming the orientation of the catheter relative to the portion of the bodily passage intended to be treated;
positioning the catheter to align the raised element with the portion of the bodily passage intended to be treated by the catheter;
inflating the inflatable balloon of the catheter until the raised element contacts the tissue of the portion of the bodily passage intended to be treated by the catheter;
following the step of inflating the inflatable balloon, deflating the inflatable balloon; and
withdrawing the catheter from the bodily passage;
wherein the marking indicia is adapted to indicate the location of the raised element about the circumference of the elongate shaft.
